(19) United States
(12) Patent Application Publication
(10) Pub. No.: US 2019/0254363 A1
(43) Pub. Date: Aug. 22, 2019

(54) Coughguardz

(71) Applicant: Justin Earl Bird, Houston, TX (US)

(72) Inventor: Justin Earl Bird, Houston, TX (US)

(21) Appl. No.: 16/345,705

(22) PCT Filed: Mar. 2, 2017
(86) PCT No.: PCT/US17/20340

§ 371 Date: Apr. 27, 2019

Related U.S. Application Data
(60) Provisional application No. 62/413,523, filed on Oct. 27, 2016.

(51) Int. Cl. A41D 13/11; A41D 23/00; A61F 13/12; A61F 13/15

(52) U.S. Cl. CPC A41D 13/1161; A61F 13/15; A61F 13/122; A41D 23/00

(57) ABSTRACT

The present invention provides a device worn around the neck of an individual and comprises a concealed face mask for use in covering one's mouth to prevent the aerosolization of infectious droplets during coughing or sneezing. is disclosed. This device houses an impervious face mask that, during use, is properly positioned over the mouth and nose to most effectively trap infectious particles. When not in use, the device folds on itself, collapsing the face mask and protecting the individual's skin. The device is designed so that the individual can cover one's mouth and nose in order to contain expelled particles during coughing and sneezing without contaminating the surrounding air or the individual's hands.

### CROSS-REFERENCE TO RELATED APPLICATION

This PCT application claims benefit of U.S. provisional patent application Ser. No.

62/413,523, filed Oct. 27, 2016.

### FEDERAL FUNDING LEGEND

This invention was not created using federal funds.

### FIELD OF THE INVENTION

The present invention generally relates to a device or a system and a method to contain infectious particles that are released in the environment due to coughing, sneezing, blowing one's nose or similar means of dispersion. More specifically, the present invention is drawn to a device, namely Coughguardz and to a method comprising the use of this device that will prevent contamination of the environment by infectious particles that are released due to means of dispersion that include but are not limited to coughing, sneezing, or blowing one's nose.

### BACKGROUND OF THE INVENTION

Infectious particles, released during coughing, sneezing or blowing one's nose contaminate the air that one breathes and the articles that one touches. Non-infected individuals breathe these infected particles or touch the infected surfaces and then touch their own mucous membranes, consequently, becoming infected themselves. One of the ways to prevent the spread of these infectious particles is by covering one's nose and mouth while coughing and sneezing. Additionally, the use of handkerchiefs, tissues, scarfs, and other items to cover one's nose and mouth while coughing, sneezing or blowing one's nose neither protect one's hands from becoming contaminated nor do they adequately trap the infectious particles released due to coughing, sneezing or blowing one's nose. Thus, there is a long-standing and significant and un-met need in the art for a system or a device that can contain infectious particles released in the environment due to coughing, sneezing or blowing one's nose. The present invention fulfils this long-standing need in the art.

### SUMMARY OF REPRESENTATIVE EMBODIMENTS OF THE INVENTION

In a preferred embodiment, the present invention is directed to a device, comprising: more than one layer of fabric with a bottom fold and an opening in back of the fabric, wherein the fabric further comprises: at least one fastener to open and close the fabric around the individual's body; at least one face mask; at least one concealed face mask area designed to allow attachment of the face mask; at least one pull tab placed in the front of the fabric and attached securely at the upper level of the area that allows attachment of the face mask; an adjustable band enclosed and secured in the interior leaf of the fabric; or a combination thereof.

In another related embodiment, the adjustable band further comprises: more than one button holes placed at various lengths along the band.

In yet another related embodiment, the device is designed to be worn around an individual's neck or lower part of the head.

In still yet another embodiment, the face mask is placed in the interior front fold in the face mask area of the fabric.

In further yet another related embodiment, the face mask is attached to the face mask area by a fastener comprising Velcro, buttons, sliders or a combination thereof.

In another related embodiment, the pull tab when pulled elevates the face mask over an individual's nose and mouth or mouth alone.

In yet another related embodiment, the adjustable band is made of material that is stretchable, the material comprising elastic, rubber or both.

In still yet another related embodiment, the fastener to open and close the fabric comprises Velcro, button, slider, or a combination thereof.

In further yet another related embodiment, the face mask is impervious on the outer surface, is absorbent on the inner surface, is made with anti-bacterial materials, or a combination thereof.

In another preferred embodiment, the present invention is directed to a method to prevent dispersion of infectious particles by an individual, comprising: securing the face mask onto the base fabric on the device described herein using the fastener; placing the device around the neck of the individual; adjusting the device for a snug fit; securing the device around the neck; pulling the pull tab prior to the urge to sneeze, cough or blow the nose to elevate the fabric and the mask in such a manner that it covers the individual's nose and mouth or mouth; sneezing, coughing, blowing one's nose or a combination thereof into the face mask so that the infectious particles are contained by the face mask and the surrounding material; and pulling the pull tab down back to the resting position around the neck so that the face mask folds on itself and does not contaminate the individual's hands, clothing or neck.

In yet another related embodiment, the device is adjusted for a snug fit by using the button hole on the adjustable band on the device.

### BRIEF DESCRIPTION OF THE DRAWINGS

FIGS. 1AA-1C illustrate the embodiments of the invention described herein. FIG. 1A shows the device (#100) described herein with the elements of the device, including but not limited to a multilayered base fabric (#101), a concealed face mask attachment area (#102), an elastic band (#104), and a Velcro fastener (#106). FIG. 1B shows the device (#100) with the facemask (#105). FIG. 1C shows the device (#100) with the pull tab (#103).

FIG. 2 illustrates how the device (#100) is worn around the neck (#205) of an individual with the pull tab (#103) facing outwards.

FIG. 3 illustrates how the device (#100) appears when the pull tab is pulled up so that the face mask (#105) is positioned over the mouth (#215) and nose (#210).

### DESCRIPTION OF PREFERRED EMBODIMENTS

The present invention provides a device, namely Coughguardz, that can be worn around one's neck and used to prevent the aerosolization of infectious droplets during coughing, sneezing, blowing one's nose or a combination thereof. As soon as an individual feels the urge to cough, sneeze, blow his nose or a combination thereof, he can pull on the pull tab and lift the device into place over his mouth and nose. A concealed face mask is housed in the device and properly positioned to trap the infectious particles released during coughing, sneezing, blowing his nose or a combination thereof. The device allows containment of the infectious particles, thereby preventing the contamination of the air, nearby surfaces and one's hands. For instance, the device is designed so that an individual can cover one's mouth, nose or both to contain expelled particles during coughing, sneezing, blowing his nose or a combination thereof without contaminating the surrounding air or his hands. In a preferred embodiment, the device comprises an impervious face mask that, when in use, is properly positioned over the user's mouth or nose to most effectively trap infectious particles. In a related embodiment, the device disclosed herein, when not in use, folds on itself, collapsing the face mask and protecting the user's skin. In another related embodiment, the device is worn around the neck. In yet another related embodiment, the face mask in the device is concealed.

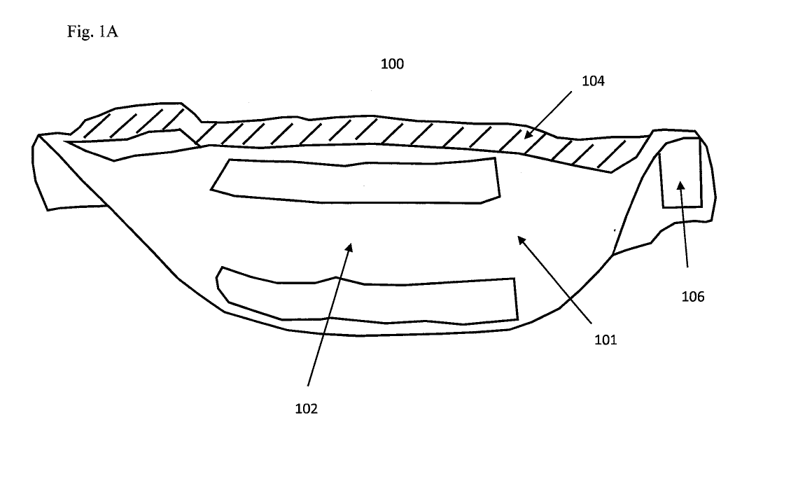

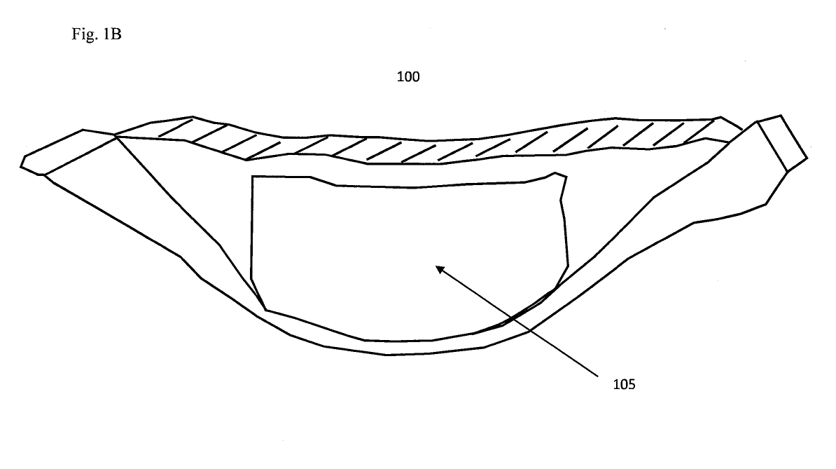

As shown in FIGS. 1A-1C, the device disclosed herein comprises multiple elements or components. For instance, the device (#100) comprises: a multilayer fabric worn around one's neck with a bottom fold and an opening in the back (#101) with a fastener (#106) that can open or close the fabric around the user's neck, at least one concealed face mask area (#102) designed to allow attachment of an impervious, absorbable face mask (#105), at least one pull tab (#103) placed on the front of the fabric that is sewn in at the upper level of the face mask attachment area (#102), an adjustable elastic band (#104) sewn into the interior leaf of the fabric. In a related embodiment, the fastener that can open or close the fabric comprises Velcro, buttons, sliders, zippers, or a combination thereof, or similar types of fasteners. In another related embodiment, the face mask has dual properties that include but are not limited to being impervious on the outer surface and absorbent on the inner surface. For instance, the absorbent layer of the face mask attracts the moisture expelled and the impervious outer layer keeps the moisture from penetrating the exterior fabric. In yet another related embodiment, the face mask is made with anti-bacterial materials. Various anti-bacterial materials can be used.

In another related embodiment, all the components disclosed above are attached to the base multilayer fabric (#101) as shown in FIGS. 1A-1C. In yet another related embodiment, the concealed face mask area (#102) is located on the interior front fold. In still yet another related embodiment, the face mask (#105) is placed in the face mask area (#102) and attached to the fabric (#101) by fasteners that include but are not limited to Velcro, snaps, or both. In yet another related embodiment, the pull tab (#103) is placed on the opposite side of the face mask area (#102) and on the exterior front fold of the fabric (#101). In still yet another embodiment, the pull tab (#103) is sewn in place into the upper side of the face mask area (#102) so that the pull tab (#103) directly elevates the face mask area (#102) when pulled. This allows proper placing of the face mask (#105) over the nose and the mouth. In another related embodiment, the elastic band (#104) is sewn into the top of the back fold so that the fold of fabric (#101) closest to the user's neck can be adjusted to the size of the user's neck for comfort and proper containment of the particles expelled during coughing or sneezing. In yet another related embodiment, the fabric (#101) also comprises the fastener (#106) to secure the device around the neck of the user, where the fastener includes but is not limited to Velcro, buttons, sliders, zippers, or a combination thereof, or a similar kind of fastener.

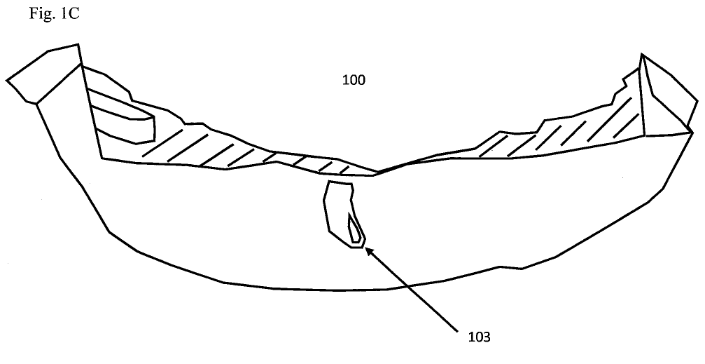

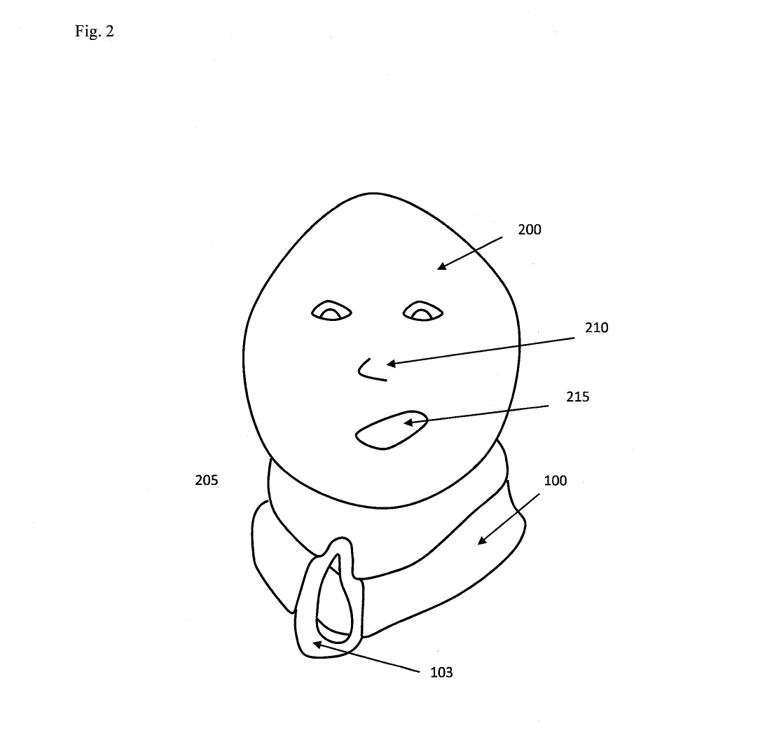

FIG. 2 illustrates one of the preferred manners in which the device (#100) described herein can be worn around the neck (#205) of an individual. This figure shows the head (#200), neck (#205), nose (#210) and mouth (#215) of an individual. The pull tab (#103) is located on the exterior front fold of the fabric (#101) and can be used to elevate the face mask area (#102) so that the face mask (#105) can be placed over the nose (#210) and the mouth (#215) of the individual.

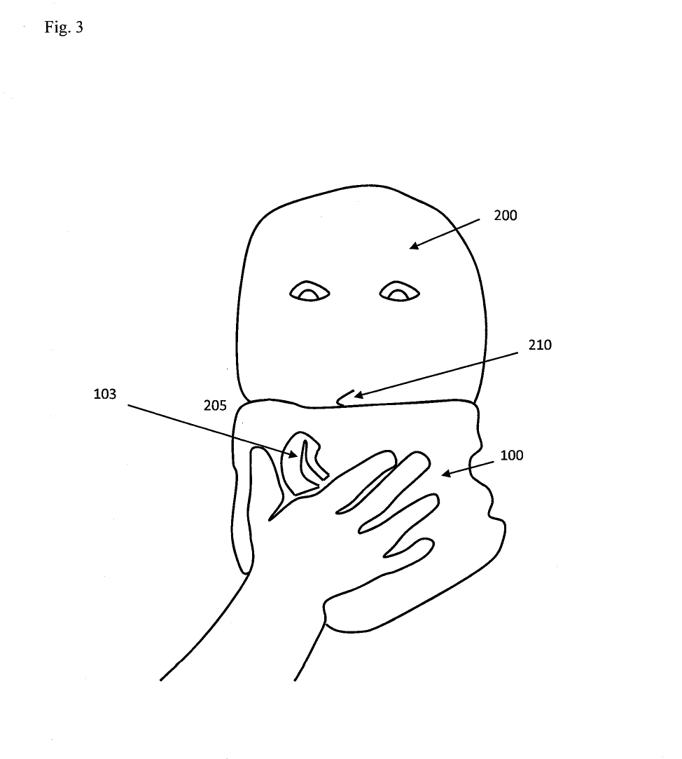

FIG. 3 illustrates one of the preferred manners in which the device (#100) described herein will appear when it is in use. For instance, the device (#100) will be pulled up to cover the nose (#210) and the mouth (#215). In another embodiment, the device (#100) may be pulled up to cover the mouth (#215) alone.

In another preferred embodiment, the present invention discloses the way the device (#100) operates. For instance, all of the components of the device (#100) are attached to the base multilayer fabric (101). The face mask area attachment area (#102) is concealed and is located on the interior front fold of the device (#100). This allows for placement of the face mask (#105). The pull tab (#103) is placed on the opposite side of the face mask attachment area (#102) on the exterior front fold of the fabric (#101). The pull tab (#103) is sewn in place into the upper region of the face mask attachment area (#102) such that when pulled, the pull tab (#103) directly elevates the face mask attachment area (#102). This properly places the face mask (#105) over the nose (#210) and mouth (#215). An elastic band (#104) is sewn into the top part of the back fold of the fabric (#101) so that the fold of the fabric (#101) closest to the user's neck (#205) can be adjusted to the size of the user's neck for comfort and proper containment of expelled particles from coughing and sneezing. The base fabric (#101) may be easily secured around the neck (#205) with fasteners (#106) that include but are not limited to Velcro, buttons or both located at the back of the base fabric (#101). The bottom fold of the base fabric (#101) creates a contained space that houses the face mask (#105) and allows the fabric (#101) to be pulled over the nose (#210) and mouth (#215). When the pull tab (#103) is used to position the face mask (#105), the face mask (#105) opens from its resting folded position. The user then coughs, sneezes, blows his nose or a combination thereof into the face mask (#105). The absorbent layer of the face mask (#105) attracts the moisture expelled and the impervious outer layer keeps the moisture from penetrating the exterior of the base fabric (#101).

In yet another preferred embodiment, the present invention discloses the method to make the device (#100). For instance, the fabric (#101) is cut into a rectangle shape. The rectangular shaped fabric (#101) is then folded, maintaining the length but halving the width. The fasteners (#106) that include but are not limited to two Velcro strips are sewn into the interior fold of the fabric (#101), centrally positioned along the length of the fabric (#101). The two Velcro strips are sewn parallel to each other at a distance equal to the distance between the top and bottom of a standard medical face mask (#105). The open edges of the fabric (#101) along its length are then sewn together. A stitch line is placed approximately 1 inch below the seam of the fabric (#101) creating a tunnel for placement of the button hole elastic band (#104). The button hole elastic band (#104) is then inserted into the tunnel and sewn into place at each end. The fabric (#101) is then folded once more, again halving the width. The Velcro strips (#106) are sewn into place at the ends (along the width) of the fabric (#101). A button (for securing the button hole elastic band at various lengths) is sewn onto the top of the interior fold of the fabric (#101) on the right-hand side approximately 2 inches from the ends with Velcro strips. The pull tab (#103) is then sewn into the exterior fold of fabric (#101) at the top of the Velcro attachment component.

In yet another preferred embodiment, the present invention discloses a method to use the device (#100). This method comprises: securing the face mask (#105) onto the base fabric (#101) using a fastener; placing the device around the user's neck (#205) and adjusting the device (#100) for a snug fit; securing the device (#100) around the neck (#205); pulling the pull tab (#103) when the user has the urge to sneeze or cough, thereby elevating the front fold of fabric (#101) and the mask (#105) to a position covering the nose (#210), mouth (#210) or both; coughing or sneezing into the face mask (#105); coughing, sneezing, blowing the user's nose or a combination thereof into the face mask (#105) so that the infectious particles released due to coughing, sneezing, blowing one's nose or a combination thereof are contained by the face mask (#105) and surrounding material; and pulling the pull tab (#103) down back to the resting position around the neck (#205) so that the face mask (#105) folds itself, such that the soiled parts of the mask (#105) are contained and do not contaminate the user's hands, clothing, neck or a combination thereof. In a related embodiment, the device is adjusted for a snug fit using means that include but is not limited to a button hole elastic feature. In yet another related embodiment, the device is secured around the neck by fastener that includes but is not limited to Velcro, button, zipper, slider, or similar kind of fastener.

As used herein, the term “fastener” refers to any means that is used to join something together. For instance, the device (#100) described herein comprises fastener that include but is not limited to Velcro, button, zipper, slider, or a combination thereof or similar kind of fastener.

The foregoing descriptions of the embodiments of the present invention have been presented for purposes of illustration and description. They are not intended to be exhaustive or to limit the present invention to the precise forms disclosed. The exemplary embodiments were chosen and described in order to best explain the principles of the present invention and its practical application, to thereby enable others skilled in the art to best utilize the present invention.

What is claimed is:

1. A device, comprising:
more than one layer of fabric with a bottom fold and an opening in back of the fabric, wherein said fabric further comprises:
at least one fastener to open and close the fabric around the individual's body;
at least one face mask;
at least one concealed face mask area designed to allow attachment of the face mask;
at least one pull tab placed in the front of the fabric and attached securely at the upper level of the area that allows attachment of the face mask;
an adjustable band enclosed and secured in the interior leaf of the fabric;
or a combination thereof.

2. The device of claim 1, wherein the adjustable band further comprises:
more than one button holes placed at various lengths along the band.

3. The device of claim 1, wherein said device is designed to be worn around an individual's neck or lower part of the head.

4. The device of claim 1, wherein the face mask is placed in the interior front fold in the face mask area of the fabric.

5. The device of claim 4, wherein the face mask is attached to the face mask area by a fastener comprising Velcro, buttons, sliders or a combination thereof.

6. The device of claim 1, wherein the pull tab when pulled elevates the face mask over an individual's nose and mouth or mouth alone.

7. The device of claim 1, wherein the adjustable band is made of material that is stretchable, said material comprising elastic, rubber or both.

8. The device of claim 1, wherein the fastener to open and close the fabric comprises Velcro, button, slider, or a combination thereof.

9. The device of claim 1, wherein the face mask is impervious on the outer surface, is absorbent on the inner surface, is made with anti-bacterial materials, or a combination thereof.

10. A method to prevent dispersion of infectious particles by an individual, comprising:
securing the face mask onto the base fabric on the device of claim 1 using the fastener;
placing the device around the neck of the individual;
adjusting the device for a snug fit;
securing the device around the neck;
pulling the pull tab prior to the urge to sneeze, cough or blow the nose to elevate the fabric and the mask in such a manner that it covers the individual's nose and mouth or mouth;
sneezing, coughing, blowing one's nose or a combination thereof into the face mask so that the infectious particles are contained by the face mask and the surrounding material; and
pulling the pull tab down back to the resting position around the neck so that the face mask folds on itself and does not contaminate the individual's hands, clothing or neck.

11. The method of claim 10, wherein the device is adjusted for a snug fit by using the button hole on the adjustable band on the device.
